(19) United States
(12) Patent Application Publication
(10) Pub. No.: US 2025/0332038 A1
(43) Pub. Date: Oct. 30, 2025

(54) ABSORBENT ARTICLE WITH EMBOSSED SHAPING ELEMENTS

(71) Applicant: Essity Hygiene and Health Aktiebolag, Göteborg (SE)

(72) Inventors: Carin HÅKANSSON, Göteborg (SE); Angelica BURVALL, Göteborg (SE); Pia KALENTUN, Göteborg (SE); Moa HOLMSTRÖM, Malmö (SE)

(73) Assignee: Essity Hygiene and Health Aktiebolag, Göteborg (SE)

(21) Appl. No.: 18/861,023

(22) PCT Filed: Jun. 22, 2022
(86) PCT No.: PCT/EP2022/066949

§ 371 Date: Oct. 28, 2024

(51) Int. Cl. A61F 13/472; A61F 13/475; A61F 13/51; A61F 13/511; A61F 13/53; A61F 13/531

(52) U.S. Cl. CPC A61F 13/472; A61F 13/4752; A61F 13/4758; A61F 13/51104; A61F 13/531; A61F 2013/51078; A61F 2013/530481; A61F 2013/5315

(57) ABSTRACT

An absorbent article includes a fluid permeable topsheet, a backsheet and an absorbent core. The absorbent core includes a first absorbent structure located between the topsheet and a second absorbent structure, the second absorbent structure being located between the first absorbent structure and the backsheet. The second absorbent structure includes a central embossed shaping element including first and second central embossed shaping portions. The first and second central embossed shaping portions extend between a longitudinal centerline and first and second side edges, respectively. An outer embossed shaping element is provided in at least either of the topsheet or the first absorbent structure. The outer embossed shaping element includes a first and a second outer embossed shaping portion. The first and second outer embossed shaping portions are, in a transversal direction, provided outwards with respect to the first central embossed shaping portion and the second central embossed shaping portion, respectively.

### TECHNICAL FIELD

The present disclosure pertains to an absorbent article for a female user in the form of a sanitary napkin, incontinence pad or liner having an absorbent core provided with embossed shaping elements to promote a good fit and leakage security.

### BACKGROUND OF THE INVENTION

Sanitary napkins and incontinence pads to which this disclosure relates are typically intended to absorb body liquids, such as urine, menses and/or discharge from the woman's lower abdomen. Such articles may be absorbent pads for heavier bladder leakage, thinner liners for light bladder leakage or sanitary napkins for menses and other body discharges.

Absorbent articles are generally provided with an absorbent core to receive and retain body liquids. For incontinence pads intended for heavier bladder leakage to function efficiently, the absorbent core must quickly acquire body liquids into the structure from the point of application and subsequently distribute the body liquids within and throughout the absorbent core to provide maximum leakage containment. It is also desirable that such pads are comfortable and have a good fit, i.e., to be sized and configured to fit the limited space available in the crotch portion of the underwear while providing the wearer with a feeling of security. Additionally, there is desire that such articles are discreet, thin, and not visible even if the user wears tight fitting, which may be seen as being in contradiction with the above objects.

It is furthermore of importance that the absorbent article, such as for example an incontinence pad, gives the user a visual perception of leakage security and good fit, enabling the user to feel secure and thereby enhancing the well-being of the user.

In view of the above, there is a need for an absorbent article for female users, which does not leak, has good fit, is discreet to wear and also gives the user a visual perception of leakage security and good fit.

### SUMMARY OF THE INVENTION

One or more of the above objects is achieved with an absorbent article for a female user according to claim 1. Further advantages and advantageous features of the invention are disclosed in the following description and in the dependent claims.

An absorbent article for a female user in the form of sanitary napkin, incontinence pad or liner disclosed herein has a first and a second longitudinal side edge and a front and a rear end edge. The absorbent article comprises a fluid permeable topsheet, a backsheet and an absorbent core being located between the topsheet and the backsheet. The absorbent article is extending in a longitudinal direction L, a transverse direction T and a thickness direction. The absorbent core being divided in a front half and a rear half and comprising a first absorbent structure and a second absorbent structure, the first absorbent structure is located between the topsheet and the second absorbent structure, and the second absorbent structure is located between the first absorbent structure and the backsheet. The second absorbent structure comprising a central embossed shaping element comprising a first central embossed shaping portion and a second central embossed shaping portion extending in the longitudinal direction at least in the front half of the absorbent core. The first central embossed shaping portion extending between a longitudinal centerline Lc and the first longitudinal side edge and the second central embossed shaping portion extending between the longitudinal centerline Lc and the second longitudinal side edge. The first and the second central embossed shaping portions converging in the direction from the front end towards the rear end of the absorbent article, and an outer embossed shaping element is provided in the topsheet and/or the first absorbent structure. The outer embossed shaping element comprising a first outer embossed shaping portion and a second outer embossed shaping portion extending in the longitudinal direction of the absorbent article. The first outer embossed shaping portion is, in the transversal direction, provided outwards with respect to the first central embossed shaping portion, and the second outer embossed shaping portion is, in the transversal direction, provided outwards with respect to the second central embossed shaping portion. So, the first outer embossed shaping portion is extending between the first central embossed shaping portion and the first longitudinal side edge, and the second outer embossed shaping portion is extending between the second central embossed portion and the second longitudinal side edge. The first and second outer embossed shaping portions are also converging in the direction from the front end towards the rear end of the absorbent article. The outer embossed shaping element is provided in at least either of the topsheet or the first absorbent structure.

Each of the first and second outer embossed shaping portions may be formed by a continuous embossed line, or may alternatively be formed by two or more continuous embossed lines. Alternatively, the outer embossed shaping element may be formed by a continuous or discontinuous embossment pattern extending in the longitudinal direction L and on a respective side of the central embossed shaping element.

The outer embossed shaping element may also comprise print pattern to further visualize the outer embossed shaping element.

The central embossed shaping element may have a V-shape wherein the first central embossed shaping portion has a V-shaped portion first leg and the second central embossed shaping portion has a V-shaped portion second leg, and the V-shaped portion first leg and the V-shaped portion second leg may be converging in a converging point. The V-shaped embossing in the front half promotes the second absorbent structure, during use of the absorbent article to flex in the front half of the absorbent article along the V-shaped portion and to adopt a bowl like shape in the front half where the V-shaped portion first and second legs converge in a converging point.

Furthermore, the fact that the absorbent article also comprises an outer embossed shaping element provided in at least one layer located above the second absorbent structure, and that the outer embossed shaping element may comprise a first embossed line and a second embossed line, each extending in the longitudinal direction and on a respective side of the central embossed shaping element and thus matches the V-shaped embossing in the second absorbent layer promotes a predictable flexing and bowl shaping of the absorbent article in the front half where the inlet zone is located. As first and second outer embossed portions of the outer embossed shaping element are provided on a respective side of the central embossed shaping element, the outer embossed shaping element will cooperate with the central embossed shaping element with respect to the flexing to make a bowl shape.

The outer embossed shaping element may be provided in the topsheet and the first absorbent structure. The first absorbent structure may also comprise a fluid control layer and/or a first absorbent layer, and the outer embossed shaping element may be provided in the topsheet, the fluid control layer and/or the first absorbent layer. Absorbent articles, such as for example incontinence pads intended for heavier bladder leakage may include a multi absorbent layer construction, with the bottom absorbent core, i e the second absorbent structure, intended for storing a larger amount of liquid. According to one embodiment constitutes the first absorbent structure only a fluid control layer and the outer embossed shaping element is provided in the topsheet and the fluid control layer. The fluid control layer may be located between the topsheet and the first absorbent layer, or alternatively, between the first absorbent layer and the second absorbent structure. According to this embodiment may the outer embossed shaping element be provided in the topsheet and the layer closest to the topsheet of either the fluid control layer or the first absorbent layer. The outer embossed shaping element may also be provided in the topsheet, the fluid control layer and the first absorbent layer. According to a further embodiment may the first absorbent structure constitute only a first absorbent layer.

The first and the second outer embossed shaping portions converging towards the rear half of the absorbent article and each of the first and second outer embossed shaping portions extending in the longitudinal direction and on a respective side of the central embossed shaping element. The first and the second outer embossed shaping portions of the outer embossed shaping element may converge in relation to each other towards the rear end edge of the absorbent article.

The outer embossed shaping element may further comprise an arc-shaped embossed front portion arranged frontwards of the central embossed shaping element and substantially following the contour of the V-shaped or modified V-shaped portion of the central embossed shaping element. The arc-shaped embossed front portion may together with the central shaping element promote an enhanced and predictable flexing to make a bowl shape.

The central embossed shaping element may also comprise an I-shaped portion, the I-shaped portion may extend along the longitudinal centerline Lc from the converging point towards the rear end. The I-shaped portion of the central embossed shaping element may be a continuous or a discontinuous I-shaped portion. The first outer embossed shaping portion and the second outer embossed shaping portion may each further extend in the longitudinal direction on a respective side of the I-shaped portion of the central embossed shaping element, optionally up to or past a rear end point of the I-shaped portion of the central embossed shaping element.

The first and the second outer embossed shaping portions of the outer embossed element may extend on a respective side of the longitudinal centerline Lc and the transversal distance between the first and the second outer embossed shaping portion in a rear third of the absorbent core may be within the range of from 0.5 to 20 mm. This has been found to cooperate with the central embossed shaping element and improve the prediction of the shaping also in the rear part of the absorbent article so the article aligns with and fit comfortably into the crease-like regions of the buttocks and perineum. The first outer embossed portion of the outer embossed shaping element may be concave as seen from the longitudinal centerline Lc and the second outer embossed portion of the outer embossed shaping element may also be concave as seen from the longitudinal centerline Lc.

The second absorbent structure may comprise one second absorbent layer. The second absorbent structure may comprise superabsorbent polymers in an amount of from 30 wt. %, optionally from 35 wt % or optionally from 40 wt. % based on the total second absorbent layer, such as within the range of from 30 wt %, 35 wt % or 40 wt. % to 80 wt. %. Due to a higher content of superabsorbent polymers in the second absorbent structure, the core structure may be perceived as less flexible and less prone to shape in response to the user body. However, such article may become difficult to shape in a predictable manner. The central embossed shaping element and the outer embossed shaping element however enables an improved flexing and shaping of such absorbent article for female users.

The second absorbent layer may have a higher density than the first absorbent structure. The first absorbent structure may comprise a fluid control layer and/or a first absorbent layer and the second absorbent structure may have a higher density than the fluid control layer and the first absorbent layer. The second absorbent layer may have at least 50%, such as 75% or 100% higher density than each of the fluid control layer and the first absorbent layer. The second absorbent structure may be between 120 kg/m³ and 500 kg/m³, optionally between 135 kg/m³ and 350 kg/m³ and optionally between 150 kg/m³ and 350 kg/m³. Due to a relatively high density in the second absorbent structure and to a relatively high content of superabsorbent polymers, the core structure may be perceived as less flexible and less prone to shape in response to the users body. However, the central embossed shaping element and the outer embossed shaping element in the front half enables an improved flexing and promote the absorbent core, during use of the absorbent article to flex and to adopt a bowl like shape in the front half.

The second absorbent structure may comprise a first side cut along the first longitudinal side edge and a second side cut along the second longitudinal side edge so as to create a transversal narrower width where the side cuts are arranged. The first and the second side cuts may be arranged on either side of and aligned with a converging point of the V-shaped portion first leg and the V-shaped portion second leg, or of an extension of the V-shaped portion first leg and the V-shaped portion second leg, i.e., for non-intersecting V-shaped portion first and second legs. The second absorbent structure may be wrapped in a core wrap, for example of tissue or nonwoven. The side cuts cooperate with the central embossing element to shape the pad in a desired and predictable manner. The side cuts may render the second absorbent structure more flexible and thus more comfortable for the user. The side cuts promote the second absorbent structure, during use of the absorbent article to flex and to adopt a bowl like shape in the front half where the inlet zone is located and where the V-shaped portion first and second legs converge in the converging point.

The second absorbent structure comprises at least one absorbent layer, a second absorbent layer, but may also comprise more than one layer. The central embossed element is provided in the second absorbent structure, at least in a second absorbent layer in the second absorbent structure. Side cuts may be provided in the second absorbent structure, at least in one absorbent layer in the second absorbent structure.

A ratio of a width at the narrowest section of the second absorbent structure at the first and the second side cuts and a width of the absorbent article at the same location may be from 0.5:1 to 0.8:1. This has been found providing an enhanced cooperation with the central embossing shaping elements for providing the pad in a desired and predictable manner.

The absorbent core has a front part, a rear part and an intermediate part located between the front part and the rear part, and the widest transversal width of the absorbent core may be in the front part. A transition width (W₈) between the front part and the intermediate part of the absorbent core may be narrower than the transversal width of the rest of the absorbent core. The second absorbent structure may have a narrower transversal width W₁₃ between the front part and the intermediate part of the absorbent core than the transversal width of the absorbent core W₈. The transversal width of the second absorbent structure, W₁₃, may be narrower than the transversal width of the first absorbent layer between the front part and the intermediate part of the absorbent core. The front part, the rear part and the intermediate part of the absorbent core may be of substantially equal length. The central embossed shaping element may be extending in the front part and in the intermediate part of the absorbent core. The outer embossed shaping element may be extending in the front part and in the intermediate part of the absorbent core. The outer embossed shaping element may also extend in part of the rear part of the absorbent core, and according to this embodiment has the outer embossed shaping element a greater extension in the longitudinal direction in the rear half than the central embossed shaping element. The outer embossed shaping element may have a greater extension in the longitudinal direction, also in the front part, towards the front end, than the central embossed shaping element.

A converging point of a V-shaped central embossed shaping element may be between the front part and the intermediate part of the absorbent core where the transversal width may be narrower than the transversal width of the rest of the absorbent core. The V-shaped embossing in the front part promotes the absorbent core, during use of the absorbent article to adopt a bowl like shape in the front part where a V-shaped portion first and second legs converge in a converging point.

### BRIEF DESCRIPTION OF THE DRAWINGS

The present invention will be further explained hereinafter by means of non-limiting examples and with reference to the appended drawings wherein:

FIG. 1 shows a top plan view of an incontinence pad as disclosed herein;

FIG. 2 shows an exploded view of the incontinence pad in FIG. 1; and

FIG. 3 shows the incontinence pad from FIG. 1, flexed and shaped as during use.

### DETAILED DESCRIPTION

The invention will be described more closely below by reference to an exemplary embodiment. The invention may however be embodied in many different forms and should not be construed as limited to the embodiments set forth in the drawings and the description thereto.

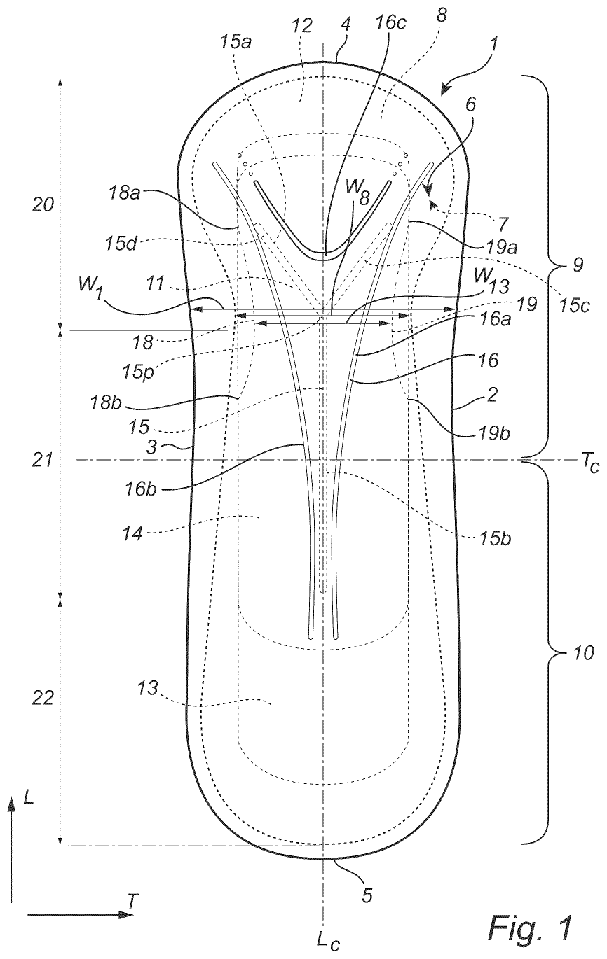

FIGS. 1 to 3 show an incontinence pad 1 according to the present disclosure. FIG. 1 schematically shows the incontinence pad 1 seen from the side that is intended to be facing towards a wearer's body when the pad 1 is being worn.

The incontinence pad 1 has a first and a second longitudinal side edge 2, 3 and a front and a rear end edge 4, 5. The pad 1 comprises a fluid permeable topsheet 6, a backsheet 7 and an absorbent core 8 being located between the topsheet 6 and the backsheet 7. The incontinence pad 1 extends in a longitudinal direction L and a transverse direction T and the absorbent core 8 is divided in a front half 9 and a rear half 10 and has an inlet zone positioned in the front half 9. The absorbent core 8 comprises a first absorbent layer 12, a second absorbent layer 13 and a fluid control layer 14, the first absorbent layer 12 being arranged between the fluid control layer 14 and the second absorbent layer 13 and the fluid control layer 14 being arranged between the topsheet 6 and the first absorbent layer 12.

The topsheet 6 may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The topsheet 6 may consist of non-absorbent fibers, such as thermoplastic fibers. The materials suited as topsheet material should be soft and non-irritating to the skin and be readily penetrated by body fluid, such as menstrual fluid and urine.

The backsheet 7 may comprise or consist of a thin plastic film, e.g., a polyethylene or polypropylene film, a nonwoven material coated with a liquid impervious material, a hydrophobic nonwoven, which resist liquid penetration. Laminates of plastic films and nonwoven materials may also be used. The backsheet material can be breathable to allow vapor to escape from the absorbent structure, while still preventing liquids from passing through the backsheet material.

The absorbent core 8 constitutes the absorbent structure of the article which acquires, distributes and stores bodily fluids. The absorbent core 8 comprises a first absorbent structure 11 and a second absorbent structure 13. The first absorbent structure 11 comprising at least one absorbent layer arranged between the topsheet and the second absorbent structure 13, and this absorbent layer may be a fluid control layer 14 or a first absorbent layer 12, or both a fluid control layer 14 and a first absorbent layer 12. The first absorbent structure 11 in FIG. 1-3 comprises a fluid control layer 14 and a first absorbent layer 12. The fluid control layer 14 is located between the topsheet 6 and the first absorbent layer 12. The second absorbent structure 13 is arranged between the first absorbent structure 11 and the backsheet 7 and the central embossed shaping element is provided in the second absorbent structure 13 and the outer embossed shaping 15 element 16 is provided in at least either of the topsheet 6 or the first absorbent structure 11. The outer embossed shaping element 16 in the incontinence pad 1 in FIG. 1-3 is provided in the topsheet 6, the fluid control layer 14 and the first absorbent layer 12. The second absorbent structure 13 in FIG. 1-3 comprises one second absorbent layer 13, and the central embossed element 15 is provided in the second absorbent layer 13. The first absorbent layer 12 and second absorbent structure 13 may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form suitable for use in absorbent composites including particles, fibres, flakes, spheres, and the like. A high absorption capacity is provided using high amounts of superabsorbent material. Thin absorbent cores which are common in for example sanitary napkins, baby diapers and incontinence guards, often comprise a compressed, mixed or layered structure of cellulosic fluff pulp and superabsorbent polymers. The second absorbent layer 13 may comprise superabsorbent polymers in an amount of from 30 wt. %, optionally in an amount of from 30 wt. % to 80 wt. %.

The first absorbent layer 12 may more specifically be an airlaid nonwoven layer or a matformed absorbent layer. The density of the first absorbent layer 12 may be within the range of from 20 to 120 kg/m³. The second absorbent structure 13 in the incontinence pad 1 is one layer, the second absorbent layer 13. The second absorbent layer 13 may comprise superabsorbent polymers in an amount of 30 wt %, 35 wt% or 40 wt. % or more. The superabsorbent polymer may for example be mixed with cellulosic fluff pulp. The second absorbent layer 13 is optionally wrapped in a core wrap. The density of the second absorbent layer may be within the range of from 120 to 500 kg/m³. The fluid control layer 14 is adapted to quickly receive and temporarily store discharged liquid before it is absorbed by the first and second absorbent layers 12, 13. The fluid control layer 14 may be a fibrous layer comprising or consisting of synthetic fibers and may for example be spunbond nonwoven, spunlace nonwoven or high loft nonwoven.

The second absorbent layer 13 is provided with a central embossed shaping element 15 in the form of a Y-shaped embossing comprising a V-shaped portion 15a provided in the front half 9 of the absorbent core 8 and an I-shaped portion 15b provided at least partly in the rear half 10 of the absorbent core. The V-shaped portion 15a including a V-shaped portion first leg 15c and a V-shaped portion second leg 15d converging within an inlet zone in the front half 9 of the absorbent core 8. The I-shaped portion 15b may be a continuous or a discontinuous I-shaped portion. The central embossed shaping element 15 may constitute of a continuous embossing or by discontinuous elements, such as dots, forming the central embossed shaping element 15. The central embossed shaping element 15 may alternatively be a modified Y-shaped portion comprising a modified V-shaped portion, such as with a slightly rounded tip portion or with the V-shaped portion first leg and second leg being non-intersecting.

The absorbent core has also a front part 20, a rear part 22 and an intermediate part 21 located between the front part 20 and the rear part 22, and the widest transversal width of the absorbent core 8 is in the front part 20. A transition width W₈ between the front part 20 and the intermediate part 21 of the absorbent core 8 may be narrower than the transversal width of the rest of the absorbent core 8. The second absorbent structure 13 has a narrower transversal width W₁₃ between the front part and the intermediate part of the absorbent core 8 than the transversal width of the absorbent core W₈. The transversal width of the second absorbent structure, W₁₃, is narrower than the transversal width of the first absorbent layer 12 between the front part 20 and the intermediate part 21 of the absorbent core 8. The front part 20, the rear part 22 and the intermediate part 21 of the absorbent core 8 are of substantially equal length.

A converging point 15p of the V-shaped central embossed shaping element 15 is between the front part 20 and the intermediate part 21 of the absorbent core 8 where the transversal width is narrower than the transversal width of the rest of the absorbent core 8. The V-shaped embossing in the front part 20 promotes the absorbent core 8, during use of the incontinence pad 1 to adopt a bowl like shape in the front part 20 where a V-shaped portion first and second legs 15c, 15d converge in a converging point 15p.

The incontinence pad 1 has also an outer embossed shaping element 16 provided in the topsheet 6, the fluid control layer 14 and the first absorbent layer 12, the outer embossed shaping element 16 comprising a first embossed line 16a and a second embossed line 16b, each extending in the longitudinal direction L and on a respective side of the central embossed shaping element 15, as seen in the transverse direction T. The outer embossed shaping element 16 is extending in the front part 20 and in the intermediate part of the absorbent core. The first and the second embossed lines 16a, 16b of the outer embossed shaping element 16 converge in the direction towards the rear end edge 5 of the incontinence pad 1. In the FIGS. 1-3, the outer embossed shaping element 16 further comprises an arc-shaped embossed front portion 16c arranged frontwards of the Y central embossed shaping element 15 and substantially following the contour of the V-shaped or modified V-shaped portion 15a of the central embossed shaping element 15. The first and the second embossed lines 16a, 16b of the outer embossed element 16 extend on a respective side of a longitudinal centerline Lc of the incontinence pad 1 and are provided with a distance to each other in the rear half 10, as measured in the transverse direction T being within the range of 5 to 15 mm or 0.5 to 20 mm.

The second absorbent layer 13 comprises a first side cut 19 along the first longitudinal side edge 2 and a second side cut 20 along the second longitudinal side edge 3. The first and the second side cuts 19, 20 are arranged on either side of and aligned with a converging point 15p of the V-shaped portion first leg 15c and the V-shaped portion second leg 15d, or of an extension of non-intersecting V-shaped portion first and second legs.

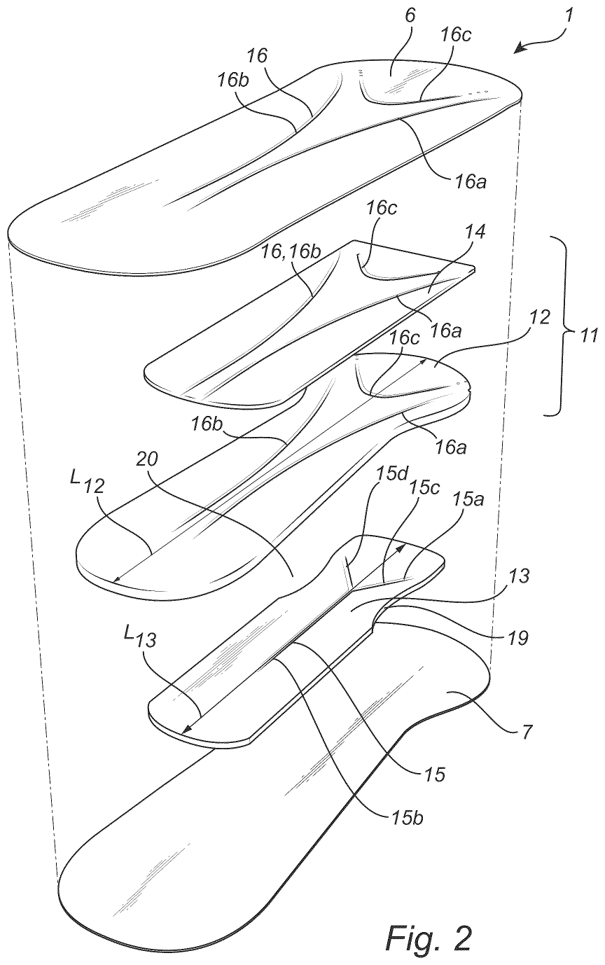

A ratio of a width W₁₃ at the narrowest section of the second absorbent layer 13 at the first and the second side cuts 19, 20 and a width W₁ of the incontinence pad 1 at the same location is from 0.5:1 to 0.8:1. As illustrated in FIGS. 1 and 2, the width of the second absorbent layer 13 is relatively constant, except at the first and second side cuts 19, 20.

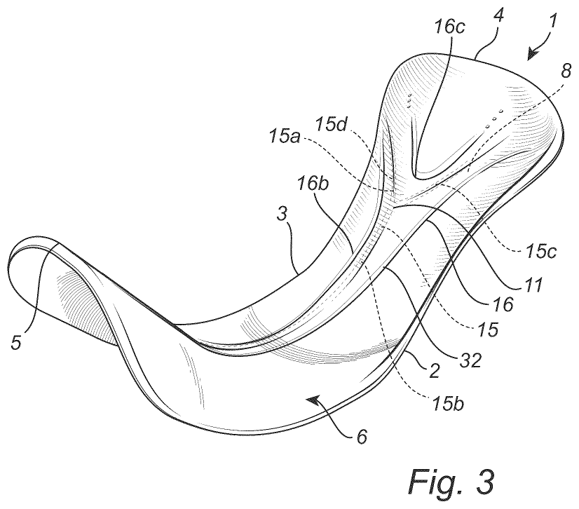

FIG. 3 illustrates the incontinence pad 1 from FIGS. 1 and 2 when being flexed and shaped as during use. The disclosed configuration of the incontinence pad 1 with a combination of a central embossed shaping element 15 in a second absorbent structure 13 below a first absorbent structure with an outer embossed shaping element enables an improved and predictable shaping during use of the incontinence pad 1. During use, the incontinence pad 1 closely adopt to the female anatomy and allows the front half of the article to fit over and conform to the flattened forward portion of the female genitalia and to form a front bowl shape for acquiring the body liquids, while the rear half of the article aligns with and fit comfortable into the crease-like regions of the buttocks and perineum. Furthermore, the arc-shaped embossed front portion 16c arranged frontwards of the Y central embossed shaping element 15 and substantially following the contour of the V-shaped or modified V-shaped portion 15a of the central embossed shaping element 15, promotes an enhanced bowl shape around the inlet zone in the front half 9.

### Thickness Measurement

To determine the density of the first and the second absorbent structure is the thickness determined by means of a measuring foot with affixed load of 0.5 k Pa. The foot is lowered onto the absorbent layer at a rate of 13 mm/s. The thickness is read off at the digital thickness gauge/tester after 5 seconds when the measuring foot has touched the surface of the absorbent structure.

1. An absorbent article for a female user in the form of a sanitary napkin, incontinence pad or liner having a first and a second longitudinal side edge and a front and rear end edge, and comprising a fluid permeable topsheet, a backsheet and an absorbent core being located between the topsheet and the backsheet, the absorbent article is extending in a longitudinal direction, a transverse direction and a thickness direction, the absorbent core being divided in a front half and a rear half and comprising a first absorbent structure and a second absorbent structure, the first absorbent structure is located between the topsheet and the second absorbent structure, and the second absorbent structure is located between the first absorbent structure and the backsheet, wherein the second absorbent structure comprises a central embossed shaping element comprising a first central embossed shaping portion and a second central embossed shaping portion extending in the longitudinal direction in the front half of the absorbent core, the first central embossed shaping portion extending between a longitudinal centerline Lc and the first longitudinal side edge and the second central embossed shaping portion extending between a longitudinal centerline Lc and the second longitudinal side edge, the first and second central embossed shaping portions converging in the direction from the front end towards the rear end, and an outer embossed shaping element is provided in topsheet and/or the first absorbent structure, the outer embossed shaping element comprising a first outer embossed shaping portion and a second outer embossed shaping portion extending in the longitudinal direction of the absorbent article and the first outer embossed shaping portion is, in the transversal direction, provided outwards with respect to the first central embossed shaping portion, and the second outer embossed shaping portion is, in the transversal direction, provided outwards with respect to the second central embossed shaping portion.

2. The absorbent article according to claim 1, wherein the central embossed shaping element has a V-shape wherein the first central embossed shaping portion is a V-shaped portion first leg and the second central embossed shaping portion is a V-shaped portion second leg, and the V-shaped portion first leg and the V-shaped portion second leg is converging in a converging point.

3. The absorbent article according to claim 1, wherein the outer embossed shaping element is provided in the topsheet and the first absorbent structure.

4. The absorbent article according to claim 1, wherein the first absorbent structure comprising a fluid control layer and/or a first absorbent layer and the outer embossed shaping element is provided in the topsheet, the fluid control layer and/or the first absorbent layer.

5. The absorbent article according to claim 1, wherein the outer embossed shaping element further comprises an arc-shaped embossed front portion arranged frontwards of the central embossed shaping element and substantially following the contour of the V-shaped or modified V-shaped portion of the central embossed shaping element.

6. The absorbent article according to claim 2, wherein the central embossed shaping element also comprises an I-shaped portion, the I-shaped portion extending along the central longitudinal axis from the converging point towards the rear end.

7. The absorbent article according to claim 6, wherein the first outer embossed shaping portion and the second outer embossed shaping portion, each extend in the longitudinal direction on a respective side of the I-shaped portion of the central embossed shaping element.

8. The absorbent article according to claim 1, wherein the first and the second outer embossed shaping portions of the outer embossed element extend on a respective side of a longitudinal centerline of the absorbent article and wherein a distance between the first and the second embossed element in a rear third of the absorbent core is within the range of from 0.5 to 20 mm.

9. The absorbent article according to claim 1, wherein the first outer embossed shaping portion of the outer embossed shaping element is concave as seen from the longitudinal centerline and the second outer embossed shaping portion of the outer embossed shaping element also is concave as seen from the longitudinal centerline.

10. The absorbent article according to claim 1, wherein the second absorbent structure comprises superabsorbent polymers in an amount of from 30 wt. %.

11. The absorbent article according to claim 1, wherein the absorbent core has a front part, a rear part and an intermediate part located between the front part and the rear part, wherein the widest transversal width of the absorbent core is in the front part.

12. The absorbent article according to claim 11, wherein a transition width between the front part and the intermediate part of the absorbent core being narrower than the transversal width of the rest of the absorbent core.

13. An The absorbent article according to claim 12, wherein a transition width of the second absorbent structure between the front part and the intermediate part of the absorbent core is narrower than a transition width of the absorbent core between the front part and the intermediate part of the absorbent core.

14. The absorbent article according to claim 2, wherein the second absorbent structure comprises a first side cut along the first longitudinal side edge and a second side cut along the second longitudinal side edge and wherein the first and the second side cuts are arranged on either side of and aligned with a converging point of the V-shaped portion first leg and the V-shaped portion second leg, or of an extension of a non-intersecting V-shaped portion first leg and the V-shaped portion second leg.

15. The absorbent article according to claim 1, wherein the second absorbent structure is wrapped in a core wrap.
